(19) United States
(12) Patent Application Publication
(10) Pub. No.: US 2006/0246535 A1
(43) Pub. Date: Nov. 2, 2006

(54) Agglutination tests for detection of microorganisms

(76) Inventors: Edward R. Burns, Fresh Meadows, NY (US); John C. McKitrick, Brewster, NY (US); Ding Wen Wu, Franklin Square, NY (US)

Correspondence Address: AMSTER, ROTHSTEIN & EBENSTEIN LLP, 90 PARK AVENUE, NEW YORK, NY, 10016, US

(21) Appl. No.: 10/541,520

(22) PCT Filed: Jan. 8, 2004
(86) PCT No.: PCT/US04/00338

§ 371 Date: May 11, 2006

Related U.S. Application Data
(60) Provisional application No. 60/438,918, filed on Jan. 9, 2003.

(51) Int. Cl. C12Q 1/04

(52) U.S. Cl. 435/34

(57) ABSTRACT

Methods of detecting a microorganism in an aqueous solution or suspension, where the aqueous solution or suspension does not comprise precultured microorganisms, are provided. The methods utilize microspheres coated with antibodies or antibody fragments, and assessment of agglutination. Methods of detecting at least one of n microorganism species in an aqueous solution or suspension are also provided. The methods utilize antibody coated microspheres coated with n distinct antibodies or antibody fragments and assessment of agglutination.

### CROSS-REFERENCE TO RELATED APPLICATION

This application claims the benefit of U.S. Provisional Application No. 60/438,918, filed Jan. 9, 2003.

### BACKGROUND OF THE INVENTION

(1) Field of the Invention

The present invention generally relates to methods for detecting microorganisms in solutions or suspensions. More specifically, the invention provides agglutination methods for detecting microorganisms in solutions or suspensions.

(2) Description of the Related Art

### REFERENCES CITED

Anderson K C, Lew M A, Gorgone B C, et al: Transfusion-related sepsis after prolonged platelet storage. Am J Med 1986; 81:405-410.
Brecher M E, Booth G, Kerr A: The use of a chemiluminescence-linked universal bacterial ribosomal RNA gene probe and blood gas analysis for the rapid detection of bacterial contamination in white cell-reduced and nonreduced platelets. Transfusion 1993;33:450-457.
Center for Biologics Evaluation and Research, FDA: Workshop on bacterial contamination of platelets. Bethesda: Sep. 24, 1999. Available from http://www.fda.gov/cber/minutes/workshop-min.htm.
Engelfriet C P, Reesink H W, Blajchman M A, et al: Bacterial contamination of blood components. Vox Sang 2000;78:59-67.
Hinnebusch C J, Nikolai D M, Bruckner D A: Comparison of API Rapid STREP, Baxter-MicroScan Rapid PosID panel, BBL Minitek Differential Identification system, IDS Rapid STR system, and Vitek GPI to conventional biochemical tests for identification of viridans streptococci. Am J Clin Pathol. 1991;96:459-463.
Jafardi M, Forsberg J, Gilcher R, et al: Salmonella sepsis caused by a platelet transfusion from a donor with a pet snake. N Engl J Med 2002; 347:1075-1078.
Kuehnert, M, Roth, V R, Haley, N R, Gregory, K R, et al:Transfusion-transmitted bacterial infection the United States, 1998 through 2000. Transfusion 2001; 41:1493-1499.
McDonald et al: Preliminary study to evaluate the detection of bacteria in platelet concentrates by monitoring oxygen content. Transfusion 2001; 41(Suppl.):33S (Abstr#S117).
Rabello et al: Serodiagnosis of Trypanosoina cruzi infection using the new particle gel immunoassay—ID-PaGIA Chagas. Mem Inst Oswaldo Cruz, Rio de Janeiro 1999; 94:77-82.
Rhame F D, Root R K, MackLowery J D, et al: Salmonella septicemia from platelet transfusions. Ann Int Med 1973;78:633-641.

Rumsey D H, Ciesielski D J: New protocols inserologic testing: a review of techniques to meet today's challenges. Immunohematol. 2000; 16:130-137.
Seaver M, Crookston J C, Roselle D C, Wagner S J: First results using automated epifluorescence microscopy to detect Escherichia coli and Staphylococcus epidermidis in WBC-reduced platelet concentrates. Tansfusion 2001;41:1351-1355.
Sen K, Asher D: Multiplex PCR for detection of Enterobacteriaceae in blood. Transfusion 2001;41: 1365-1372.
Wallas C H: Transfusion of Blood components contaminated with bacteria. IN Smith D M and Dodd R Y (Eds). Transfusion Transmitted Infections ASCP Press, Chicago, Ill., 1991, pp 195-206.
Yomtovian R, Lazarus H M, Goodnough L T, et al: A prospective microbiologic surveillance program to detect and prevent the transfusion of bacterially contaminated platelets. Transfusion 1993;33:902-909.
U.S. Pat. App. No. 5,100,805.

Bacterial contamination of blood and blood products remains an uncommon but clinically devastating problem. The source of contamination most often stems from contaminating organisms introduced from the skin of the donor during venipuncture (Anderson et al., 1986) or rarely from donors with infection (Rhame et al., 1973) or asymptomatic bacteremia (Jafardi et al., 2002). The precise incidence of the problem is unclear as studies investigating the question have not clearly differentiated between contamination introduced by the study versus true contamination which can cause disease (Wallas, 1991). A recent study confirmed that bacteria-contaminated blood products represent the most frequently reported cause of transfusion related death to the FDA (Center for Biologics Evaluation and Research, 1999). That study determined that the rate of transfusion-transmitted bacteremia in events/million units was 9.98 for single donor platelets, 10.64 for pooled platelets, and 0.21 for red cell concentrates (Kuehnert et al., 2001). The same study documented 34 cases and nine deaths reported to the Centers for Disease Control in a two year period, a rate that exceeds viral agents. Another study identified various bacterial species found in platelet and red blood cell preparations (see slide presentation at http://www.cdc.gob/ncidod/hip/bacon/pwrpt/slidppt1.htm).

As donor screening already addresses the health issues which could exclude high risk donors and donor screening cannot detect phlebotomy related contamination, the only effective method for preventing transfusion transmitted bacterial infections would be a pre-transfusion screening test of individual units to exclude bacterial contamination. Numerous methods have been advanced to accomplish this including Gram staining (Engelfriet et al., 2000), culturing (Yomtovian et al., 1993), physiological and chromogenic enzymatic biochemical assays (Hinnebusch et al., 1991), epifluorescence microscopy (Seaver et al., 2001), bacterial ribosomal RNA gene probes (Brecher et al., 1993), multiplex PCR (Sen & Asher, 2001), and other less commonly employed assays (e.g., nucleic acid staining, fluorescent nucleic acid stain, chemiluminescence assays, detection of pH changes, glucose assays). Gram staining is relatively insensitive compared to the other methods. Culturing represents the gold standard, but cannot be performed immediately prior to transfusion. The other methods mentioned above are highly sensitive but time consuming or expensive in their present state of development.

Two other methods, both requiring culturing, have been recently approved by the FDA for monitoring bacterial contamination of platelet preparations. One method, the Bacterial Detection System developed by the Pall Corporation, detects oxygen generated by contaminating bacteria (McDonald et al.). Another method, the BacT/ALERT® system produced by bioMerieux, detects an increase in carbon dioxide in the culture.

Several agglutination methods have been developed and are commercially available for determining the species of bacteria that are cultured from body tissues or fluids or from other sources, but these methods were not developed to test the sample directly, without culturing the sample (see, e.g., list at http://vm.cfsan.fda.gov/˜ebam-al.html).

Based on the above, all methods developed so far for detecting biological contamination suffer from a lack of sensitivity and/or high expense and/or excessive length of time (>24 hr.) to obtain results of the assay and/or excessive difficulty in performing the assay. Thus, there is a need for rapid, sensitive assays to detect microorganism contamination of blood products and other solutions and suspensions. The instant invention addresses that need.

### SUMMARY OF THE INVENTION

The present invention is based in part on the discovery that agglutination methods utilizing the selectivity of antigen binding sites of antibodies are sensitive enough to selectively detect microorganism contaminants, without first culturing the microorganisms.

Accordingly, the invention is directed to methods of detecting a microorganism in an aqueous solution or suspension, where the aqueous solution or suspension does not comprise precultured microorganisms. The methods first comprise mixing the solution or suspension with microspheres coated with antibodies or antibody fragments comprising an antigen binding site selective for the microorganism species, creating a microsphere-solution/suspension mixture. The methods further comprise evaluating the microsphere-solution/suspension mixture for agglutination. In these methods, the presence of agglutination indicates that the solution or suspension contains the microorganism species.

The inventors have also discovered that microspheres for agglutination can be prepared that comprise antibodies that detect more than one microorganism, such that a single agglutination test can detect any of the organisms to which the antibodies bind.

Thus, the invention is also directed to methods of detecting at least one of a number n microorganism species in an aqueous solution or suspension. The methods comprise mixing the solution or suspension with microspheres coated with n distinct antibodies or antibody fragments comprising an antigen binding site. In these embodiments, each of the n distinct antibodies or antibody fragments comprising an antigen binding site is selective for the one of the n microorganism species, and microspheres coated with antibodies or antibody fragments comprising an antigen binding site selective for each of the n microorganism species is present. This creates a microsphere-solution/suspension mixture. The methods further comprise evaluating the microsphere-solution/suspension mixture for agglutination. In these methods, the presence of agglutination indicates that the solution or suspension contains at least one of the n microorganism species.

### BRIEF DESCRIPTION OF THE DRAWINGS

FIG. 1 shows two fluorescent micrographs demonstrating the binding of anti-Staphylococcus epidermidis (Staphepi) IgG (panel 1A) and antiserum (panel 1B) to S. epidermidis with FITC-goat anti-rabbit IgG.

FIG. 2 are photographs showing agglutination of S. epidermidis 12228 with latex microspheres coated with anti-Staphepi IgG. Panel 1 and 1a shows negative controls of microspheres coated with preimmune IgG+S. epidermidis, and microspheres coated with preimmune IgG only, respectively. Panels 2-6 show agglutination of S. epidermidis microspheres coated with anti-Staphepi-IgG at a ratio (volume of IgG/total volume of microsphere suspension) of 1:5 (Panel 2), 1:7 (Panel 3), 1:10 (Panel 4), 1:15 (Panel 5), and 1:20 (Panel 6). Panels 2a-6a show microspheres coated with anti-Staphepi-IgG at the analogous ratios, without addition of S. epidermidis.

FIG. 3 shows photographs demonstrating that agglutination on Murex Biotech paper can be readily observed by the naked eye down to at least 0.5 McFarland unit when latex-anti-Staphepi-IgG is mixed with S. epidermidis 12228. Panel 1: one S. epidermidis colony tested; Panel 2: 2 McFarland units tested; Panel 3: 1 McFarland units tested; Panel 4: 0.5 McFarland units tested; Panel 5: 0.25 McFarland units tested; Panel 6: no S. epidermidis control.

FIG. 4 shows photographs demonstrating that agglutination of latex beads becomes invisible to the naked eye on Murex Biotech paper when platelet product is added. Each panel is latex-anti-Staphepi-IgG, combined with the following: Panel 1: platelets plus one S. epidermidis 12228 colony; Panel 2: platelets plus two S. epidermidis 12228 colonies; Panel 3: S. epidermidis 12228 colony without platelets; Panel 4: platelets alone; Panel 5: platelets spiked with S. epidermidis 12228; Panel 6: no additions.

FIG. 5 shows micrographs at (SA) 100× magnification and (SB) 400× magnification providing examples of each agglutination scoring level. Panel 1: no agglutination; Panel 2: +; Panel 3: ++; Panel 4: +++; Panel 5: ++++.

FIG. 6 shows micrographs at (6A) 100× magnification and (6B) 400× magnification demonstrating detection of latex-anti-Staphepi-IgG agglutination of S. epidermidis spiked into platelets to at least 0.0625 McFarland units. The agglutination scoring level for 100×was the same as 400× with each treatment. Panel 1: platelets plus no S. epidermidis (minus score); Panel 2: platelets plus 0.0625 McFarland unit S. epidermidis (+score); Panel 3: platelets plus 0.125 McFarland unit S. epidermidis (+score); Panel 4: platelets plus 0.25 McFarland unit S. epidermidis (+score); Panel 5: platelets plus 0.5 McFarland unit S. epidermidis (++score); Panel 6: platelets plus 1 McFarland unit S. epidermidis (++score); Panel 7: platelets plus 2 McFarland units S. epidermidis (+++score); Panel 8: platelets plus 1 colony S. epidermidis (++++score).

### DETAILED DESCRIPTION OF THE INVENTION

The present invention is based in part on the discovery that agglutination tests can be used to detect bacterial contamination in aqueous solutions or suspensions without first culturing the solution or suspension (“preculturing”) to isolate the microorganisms therein.

Thus, the invention is directed to methods of detecting a microorganism (also referred to herein as a “target microorganism”) in an aqueous solution or suspension where the aqueous solution or suspension does not comprise precultured microorganisms. The methods first comprise mixing the solution or suspension with microspheres coated with antibodies or antibody fragments comprising an antigen binding site selective for the microorganism. This creates a microsphere-solution/suspension mixture. The method then comprises evaluating the microsphere-solution/suspension mixture for agglutination. In these methods, the presence of agglutination indicates that the solution or suspension contains the microorganism.

These methods are useful for detecting the microorganism in any solution or suspension. Nonlimiting examples include environmental sources such as sewage, streams, rivers, lakes, ground water, irrigation water, municipal water supplies, tap water, and wells; food or animal feed including extracts of solid food or feed; medical products including liquid medicines and solutions and suspensions or extracts of solid medicines; and vertebrate (including mammalian, e.g., human) body fluids and products such as urine, bile, stool, peritoneal washings, sputum, bronchial aspirate, cerebrospinal fluid, pus, blood, and extracts of those fluids; and blood products such as plasma, serum, and blood products that are substantially purified from other blood products, such as red blood cells, platelets, factor IX, factor VII, albumin, and antibodies. Blood products, particularly blood cells and platelets, are preferred, since there is an acute need for rapid tests for detecting contaminating microorganisms in these products.

As used herein, a microorganism is a microscopic prokaryotic, archeal, or eukaryotic organism that is suspected of contaminating the aqueous solution or suspension, including bacteria, fungi and parasitic species that are capable of infecting vertebrates. “Microorganism” does not include viruses. The target microorganisms that can be usefully detected using these methods must also be subject to production of an antibody or antigen binding site that is selective for the microorganism. As used herein, “selective” means the ability of the antibody or antigen binding site to noncovalently bind to the microorganism but not other related microorganisms. The antibody or antigen binding site useful for the present invention is not narrowly limited to any particular degree of selectivity, affinity or avidity to the target microorganism. The antibody or antigen binding site need only usefully distinguish the microorganism from another microorganism.

As used herein, “bacteria” includes Gram-negative, Gram-positive, and acid fast positive bacteria, mycoplasma, archebacteria, and rickettsia. It is understood that antibodies selective for any particular species of bacteria could be produced without undue experimentation. Preferred are bacteria that are known pathogenic contaminants. Non-limiting examples include Staphylococcus spp., Pseudomonas spp., Listeria spp., Enterobacteriaceae species, Vibrionaceae species, Clostridium spp., Campylobacter spp., Bacillus spp., Escherichia spp. (in particular E. coli), Sarcina spp., Flavobacterium spp., Klebsiella spp., Alcaligenes spp., Micrococcus spp., Streptococcus spp., Herellea spp., Corynebacterium spp., Mycoplasina spp., Pseudomonas spp., Citrobacter spp., Treponema spp., Salmonella spp., Serretia spp. (in particular S. marcescens), Yersinia spp. (in particular Y. enterocolitica), Legionella spp., Bartonella spp., and Brucella spp. Where the solution or suspension is a red blood cell or a platelet preparation, preferred bacteria are Staphylococcus epidermidis, Staphylococcus aureus, Corynebacterium spp., Salmonella choleraesuis, Klebsiella pneumoniae, Serratia marcescens, Bacillus cereus, a Streptococcus sp. of the viridans group, Pseudomonas fluorescens, Pseudomoias putida, Treponema palliduin, Yersinia enterocolitica, Citrobacter freundii, Escherichia coli, Enterobacter cloacae, or an α-hemolytic Streptococcus sp., which are all known contaminants of these products. A particularly preferred bacterial target for these embodiments is Staphylococcus epidermidis, which is a common contaminant of blood products, in particular platelets. See Example.

In other embodiments, the microorganism is a parasite, preferably a parasite that is known to cause human or animal disease. Non-limiting examples include Trypanosoma spp. (in particular T. cruzi), Plasmodium spp., Schlistosoma spp., Babesia spp., Toxoplasma spp., Brugia spp., Wuchereria spp., Borrelia spp., or Leishnzania spp. The skilled artisan would understand that production of selective antibodies to any of these species is routine.

In additional embodiments, the microorganism is a fungus, preferably a pathogenic fungus, for example Aspergillus spp., Blastomyces spp., Coccidioides spp. (in particular C. immitis), Candida spp., Histoplasma spp. (in particular H. capsulatum), or Fusarium spp. Selective antibodies to any particular fungus can also be produced using routine methodology.

These methods are particularly useful for evaluating stored blood products such as platelets or red blood cells, and can be used at any time during the storage period. In evaluating platelets, the methods find their greatest utility where the platelets have been stored at least five days, since a determination of no contamination at five days could allow longer platelet storage, e.g., to at least seven days. Moreover, a determination of contamination would provide evidence for the decision to discard platelets prior to dispensing for transfusion. However, the methods are also useful where the blood product has been stored for less than five days, particularly when they are utilized just before the contemplated infusion of the blood product into a patient.

The methods are useful with any type of microsphere known in the art, for example red blood cells (“hemagglutination”). Preferably, the microspheres are latex microspheres, since these are widely used in agglutination methods and techniques to bind antibodies to latex microspheres, either covalently or noncovalently, are well developed.

The microspheres can be either colored, white or opaque. In some embodiments, colored microspheres can enhance the visibility or detectability of agglutination. In other embodiments, the microspheres are fluorescent.

The microspheres useful for these methods are not narrowly limited to any particular diameter, and the diameter most useful for any particular embodiment can be determined by the skilled artisan without undue experimentation. In preferred embodiments, the microspheres are between 0.5 and 10 μm, more preferably between 0.5 and 1 μm. See, e.g., Example.

The invention methods encompass any method for binding the antibodies or antibody fragments to the microspheres, including noncovalent adsorption, covalent binding, e.g., with microspheres that comprise a conjugatable moiety such as an exposed amino or carboxy moiety, and indirect binding. Nonlimiting examples of indirect binding include covalently binding an antibody-binding moiety such as protein A or an anti-IgG antibody, then adsorbing the selective antibody to the microsphere; covalently or noncovalently binding a compound with reactive groups (e.g., polylysine) to the microsphere then covalently binding the antibody to the reactive groups; etc.

The methods can be used with any agglutination format, for example a hanging drop slide (as in the Example), or in wells of multiwell plates, such as a typical 96-well microtiter plate, where the microsphere-solution/suspension mixture is about 250 μl or less.

In these methods, agglutination of the microspheres can be assessed visually with the naked eye or through a microscope (e.g., as in Example), or using an instrument that measures light scattering, e.g., as described in U.S. Pat. App. No. 5,100,805. Considerably greater sensitivity can be achieved over naked-eye visual or microscopic methods by using such an instrument, along with associated analysis methods (Id.). In addition, either fluorescence microscopy or fluorescent light scatter detection could be employed when the microspheres are intrinsically fluorescent.

In some embodiments, the microsphere-solution/suspension mixture is applied to a gel surface, then the gel is subjected to centrifugation before the evaluation step. In these embodiments, agglutination of the microsphere-solution/suspension mixture causes substantial retardation of movement of the microspheres after centrifugation when compared to the microspheres without the solution. Thus, unagglutinated microspheres are pelleted; agglutinated microspheres are not. This is usually detected visually with the naked eye, although an instrument can also be used. See, e.g., Rumsey & Ciesielski, 2000, and Rabello et al., 1999. In these embodiments the microspheres are preferably colored, which aids in their visualization.

The present invention is not narrowly limited to any particular type of antibody or antibody fragment comprising an antigen binding site. Thus any type of antibody or fragment known in the art, including polyclonal, monoclonal, recombinant, Fab fragments, F(ab′)₂ fragments, or diabodies, can be used. The antibodies can also comprise a detectible label, such as a radioactive, fluorescent, hapten (e.g., digoxigenin), or ligand (e.g., biotin) labels, which could aid in detecting agglutination.

The antibodies can be very specific for one species of microorganism or even one genotype from a particular species (e.g., E. coli O157:H7). Alternatively, the antibodies can be broadly reactive to an entire genus, family or even order of microorganisms. The latter case is preferred where determination of general contamination, and not just contamination of a particular organism, is desired, for example when evaluating contamination of blood products. Conversely, narrower specificity is preferred where determination of a particular contaminant is desired, e.g., determination of E. coli O157:H7 in food.

The antibodies can also be produced by any known method, for example by injection into a vertebrate of a whole-cell preparation of the microorganism, or injection of a preparation of a portion (e.g., a polysaccharide, protein, or lipid preparation, such as a lipopolysaccharide) of the microorganism. Many such methods are known and routinely employed.

Methods for producing antibodies of either broad or narrow specificity are also well known in the art. For example, monoclonal antibodies produced from whole cell preparations can be screened and selected for broad or narrow specificity by determining binding of the test antibodies to a panel of cells of various species. Specific antibodies can also be made by immunization of antigenic components of an organism that tend to be specific or selective to that organism, such as particular proteins or antigenic determinants that tend to vary among proteins. A relatively non-specific antiserum can also be cross-adsorbed with cross-reactive organisms or cell or antigen preparations, eliminating that reactivity from the antiserum, thus increasing its specificity. In another non-limiting example, broad specificity can be achieved by immunization with a common antigen. A common antigen is an antigen that is common to more than one species. Examples include LPS of Gram-negative bacteria and a peptide that is synthesized using a consensus amino acid sequence derived from multiple species or isolates of, e.g., bacteria.

In some embodiments of the present invention, more than one microorganism can be assayed in a single test. These embodiments involve using a preparation comprising microspheres that are coated with more than one antibody or antibody fragment comprising an antigen binding site preparation. In these embodiments, each antibody or antibody fragment comprising an antigen binding site is selective for a different microorganism.

Thus, in these embodiments, the invention is directed to methods of detecting at least one of at least n microorganism species in an aqueous solution or suspension. The methods comprise mixing the solution or suspension with microspheres coated with n distinct antibodies or antibody fragments comprising an antigen binding site. Each of the n distinct antibodies or antibody fragments comprising an antigen binding site is selective for the one of the n microorganism species, and microspheres coated with antibodies or antibody fragments comprising an antigen binding site selective for each of the n microorganism species is present. As with the previously described methods, a microsphere-solution/suspension mixture is created. Also as with the previously described methods, the microsphere-solution/suspension mixture is evaluated for agglutination, where the presence of agglutination indicates that the solution or suspension contains at least one of the n microorganism species.

In these methods n can be 2, 3, 5, up to 10, or even 20 different species or more, where a separate antibody is used for each species. Further breadth of species detection can be achieved by using n different antibodies that have broad specificity, e.g., the ability to detect an entire genus of bacteria, as previously discussed.

The simultaneous testing of the presence of any of the species detected by the n antibodies can be achieved using a mixture of separately prepared microspheres, each microsphere coated with only one preparation of antibodies or antibody fragments (e.g., a polyclonal antibody preparation from one animal that shows a characteristic specificity, or a monoclonal antibody) such that the mixture comprises microspheres with n different specificities to microorganism species. In order to be effective in detecting each species, there must be sufficient microspheres that bind each species in the mixture for detectable agglutination to occur when the mixture is combined with a desired concentration of any one of the species. The determination of the proper mixture to achieve this depends on the affinity of the coated microspheres to each species and the concentration of microspheres present in the mixture that bind to each species. Such determinations can be made using routine experimentation.

In alternative embodiments, each microsphere is coated with more than one antibody or antibody fragment, such that an individual microsphere binds to all the microorganisms that each of the antibodies or antibody fragments bind. The proper amount of each antibody or antibody fragment that is utilized with the microspheres can be determined using routine experimentation.

These embodiments can be utilized with any of the solutions or suspensions previously described, including environmental samples and whole blood or blood products. Since it would be expected that the microspheres comprising various antibodies or antibody fragments would agglutinate as effectively as the microspheres previously described that comprise only one type of antibodies or fragments, the skilled artisan would understand that the methods in these embodiments would be useful utilizing any of the method parameters previously described, i.e., after any amount of storage of the solution or suspension, using any type of microsphere of any diameter, using noncovalent, covalent or indirect methods of binding the antibodies or fragments to the microspheres, etc. Additionally, it is within the scope of the invention that these multiple antibody/antibody fragment microbeads could be used with a bacteria that has been precultured from the substrate where contamination is suspected.

Some preferred embodiments of the invention are described in the following example. Other embodiments within the scope of the claims herein will be apparent to one skilled in the art from consideration of the specification or practice of the invention as disclosed herein. It is intended that the specification, together with the example, be considered exemplary only, with the scope and spirit of the invention being indicated by the claims which follow the example.

### EXAMPLE

### Detection of Staphylococcus epidermidis in Platelets Using Latex Agglutination Methods

Materials and Methods

Purification of Anti-Staphylococcus epidermidis Polyclonal IgG. Two strains of Staphylococcus epidermidis, 12228 and 14990 (ATCC, Manassas, Va.), were inoculated into rabbits by a commercial vendor (Animal Pharm, Healdsburg, Calif.) for production of rabbit anti-Staphlylococcus epidermidis polyclonal antiserum (anti-Staphepi-AS). The IgG fraction was subsequently purified (anti-Staphepi-IgG) by using ImmunoPure® IgG (Protein A) Purification Kit (Pierce, Rockford, Ill.) according to the vendor's instructions. IgG was also purified from the rabbit preimmune serum (Pi-IgG), with the same kit.

Indirect Fluorescent Antibody (IFA) Detection of Antibody Binding to Staphylococcus epidermidis. A colony of Staphylococcus epidermidis was taken from a cultured blood agar plate and then smeared on each albumin coated glass slide (Snowcoat X-tra, Surgipath, Richmond, Ill.), followed by brief heat-fixation at 60° C. One drop of the purified anti-Staphepi-IgG, or anti-Staphepi-AS, was added to each bacteria smear. Preimmune-IgG and preimmune serum were added to bacterial smears as controls under the same conditions. The slides were incubated in a moisture chamber for 30 minutes at 37° C. and then thoroughly rinsed with phosphate buffered saline, pH 7.2 (PBS). They were then rinsed with water. One drop of FITC-goat-anti-rabbit-IgG antibody was diluted 1:160 in PBS (Research Diagnostics, Flanders, N.J.) and added to each bacterial smear. The slides were again incubated in a moisture chamber, rinsed with PBS and then with water as mentioned above. After adding a drop of mounting media, BBL™ buffered glycerol mounting medium (Becton Dickinson Microbiology System, Spanks, Md.), each slide was covered with a coverslip. The fluorescence was immediately observed using an epi-fluorescent microscope (Olympus Optical, Tokyo, Japan) equipped with a camera.

Preparation of Latex-anti-Staphepi-IgG. Polystyrene latex microspheres PS03N/4670 (Bangs Lab, Carmel, Ind.) were diluted to 10 mg/ml in each tube with PBS. The latex suspension was then mixed with various concentrations of Staphepi-IgG. After incubation at room temperature for 3 hours, the mixture was washed and centrifuged 3 times in PBS. The microsphere pellet (latex-Staphepi-IgG) was then re-suspended in PBS with 0.05% bovine serum albumin (BSA), which is the storage buffer, at a concentration of 10 mg beads/ml. Latex-preimmune-IgG (Latex-Pi-IgG) was prepared in the same way.

Agglutination Test for Latex-anti-Staphepi-IgG. To compare the detection intensity of latex-anti-Staphepi-IgG, 1 drop of beads bound with various concentrations of anti-Staphepi-IgG were added within a circle on Murex Biotech paper. Latex-Pi-IgG was used as a negative IgG control. One colony of Staphylococcus epidermidis 12228 or 14490 was then added to the circle to mix with the beads. Five minutes later, the agglutination results were observed in comparison of the counterpart bacterial negative controls (i.e. the beads without addition of the bacteria), and arbitrarily scored using a 4+ grading system: (−, +, ++, +++, ++++). The concentration given strongest agglutination was chosen for the following experiments.

The detection sensitivity of the latex-anti-Staphepi-IgG was determined by adding one drop of the chosen beads into each circle on paper, and a drop of various concentrations of Staphylococcus epidermidis. Five minutes later, the agglutination results were observed and scored as described above.

Specificity of Latex-anti-Staphepi-IgG. The specificity of latex-anti-Staphepi-IgG was determined by mixing one drop of latex-anti-Staphepi-IgG (1:5, v/v) in circles on Murex Biotech paper, with one colony of Staphylococcus epidermidis, Staphylococcus aureus, Staphylococcus saprophytics, or Streptococcus pneumoniae. Five minutes later, the results were observed and scored.

Latex Agglutination Test for Detection of S. epidermidis in Platelets. A unit of fresh human platelet product (New York Blood Center, New York, N.Y.) was divided into aliquots of 0.2 ml per tube. Various concentrations of S. epidermidis, each in 0.2 ml, were added to the tubes and mixed well with the platelet products. One drop of the mixture from each tube was then added into a well of a hanging-drop glass slide. One drop of latex-anti-Staphepi-IgG was then added to the well and gently mixed with the S. epidermidis-spiked platelets. Five minutes later, agglutination was examined under a microscope at magnifications x100 and/or x400, and recorded using the following 4+tentative scoring criteria:

| | |
|---|---|
| [−] | forms no agglutinates |
| [+] | forms small agglutinates |
| [++++] | forms large agglutinates |
| [++] and [+++] | sizes of agglutinates between [+] and [++++]. |

Results

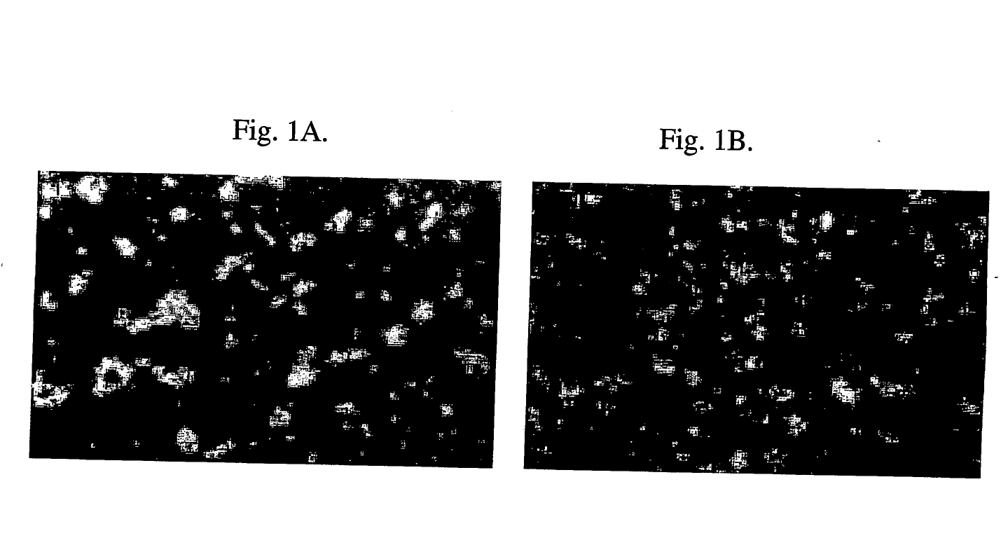

Indirect Fluorescent Antibody (IFA) Detection. Positive fluorescence was detected for the binding of anti-Staphepi-IgG or anti-Staphepi-antiserum with Staphylococcus epidermidis (FIG. 1). No fluorescence was detected from the control mixtures, preimmune-IgG or preimmune serum with Staphylococcus epidermidis, indicating no antibody binding (Table 1).

TABLE 1
Indirect Fluorescent Antibody Detection of Staphylococcus epidermidis

| | Staphylococcus epidermidis 12228 |
|---|---|
| Serum or IgG | binding |
| Preimmune serum | −^{a} |
| Preimmune IgG | − |
| anti-Staphepi-antiserum | +^{b} |
| anti-Staphepi-IgG | + |

^{a}− = negative
^{b}+ = positive

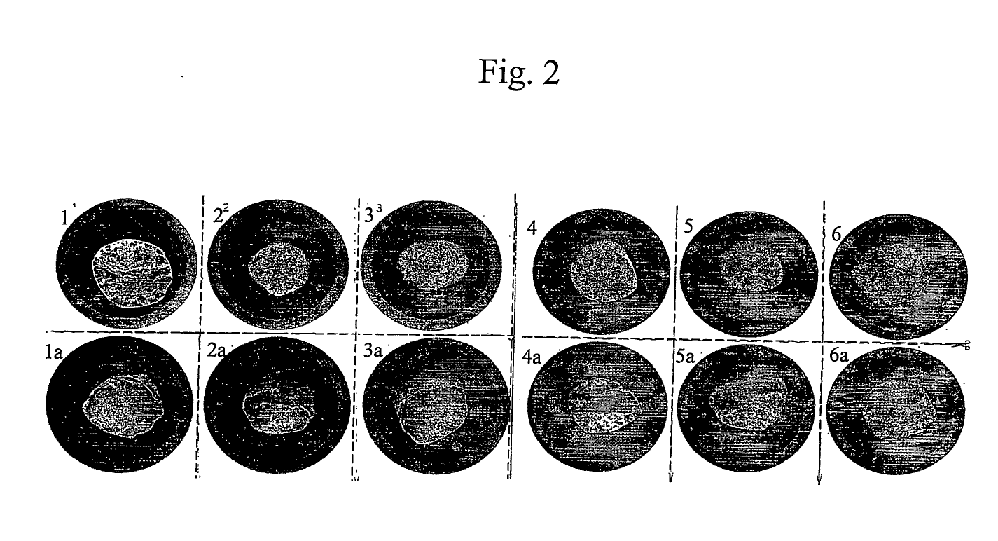

Agglutination Test for Latex-anti-Staphepi-IgG. Latex bound anti-Staphepi-IgG in the concentration (v/v, i.e. volume of IgG total volume of latex suspension) range of 1:5-1:20 detected Staphylococcus epidermidis on test paper (FIG. 2). Latex-anti-Staphepi-IgG (IgG 1:5) consistently showed the strongest agglutination for the detection, and was chosen for our other agglutination tests as further described.

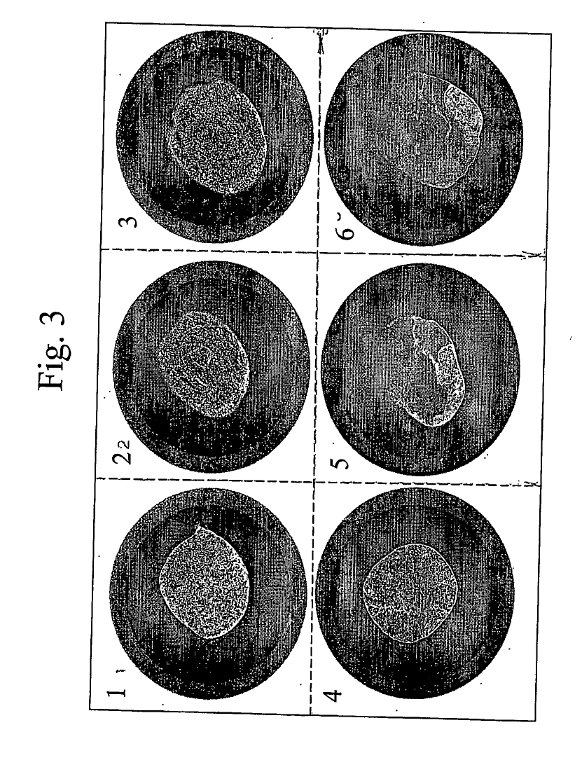

Sensitivity and Specificity of Latex-anti-Staphepi-IgG. The latex-anti-Staphepi-IgG detects Staphylococcus epidermidis in concentrations down to 0.5 McFarland unit, in a concentration-dependent manner (FIG. 3). There was marked agglutination when latex-anti-Staphepi-IgG mixed with Staphylococcus epidermidis, but no agglutination with the other Staphylococcus species, nor with Streptococcus pneumoniae (Table 2).

TABLE 2
Tests for the specificity of latex-anti-Staphepi-IgG

| | Beads | | |
|---|---|---|---|
| | Latex only | Latex- | Latex-anti- |
| | (no | preimmune- | Staphepi-I |
| Bacteria | bound IgG) | IgG^{a} | gG^{b} |
| None (Negative control) | − | − | − |
| Staphylococcus epidermidis | − | − | +++ |
| 12228 | | | |
| Staphylococcus epidermidis | − | − | +++ |
| 14990 | | | |
| Staphylococcus aureus 29213 | − | − | − |
| Staphylococcus aureus 25923 | − | − | − |
| Staphylococcus saprophytics | − | − | − |
| Streptococcus pneumoniae | − | − | − |

^{a}IgG 1:5, v/v (negative IgG control)
^{b}IgG 1:5, v/v

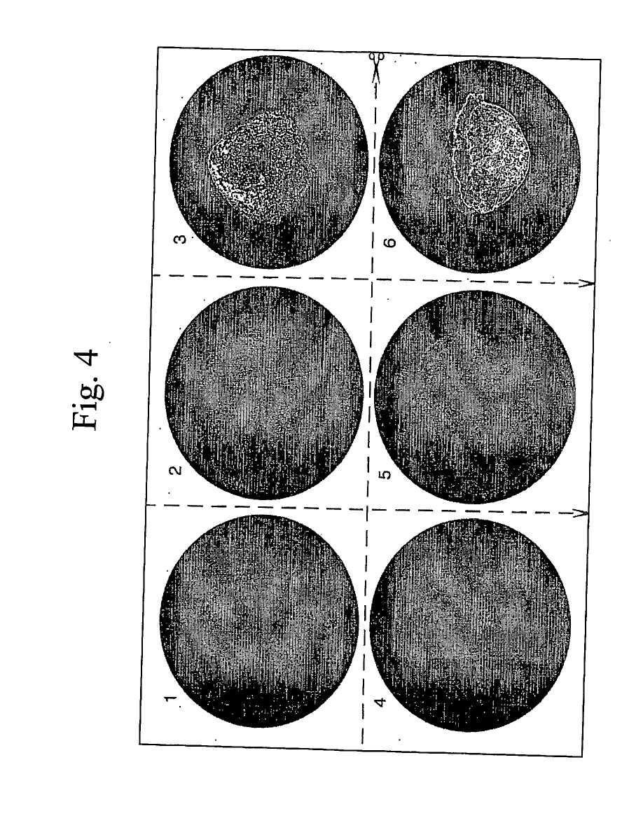

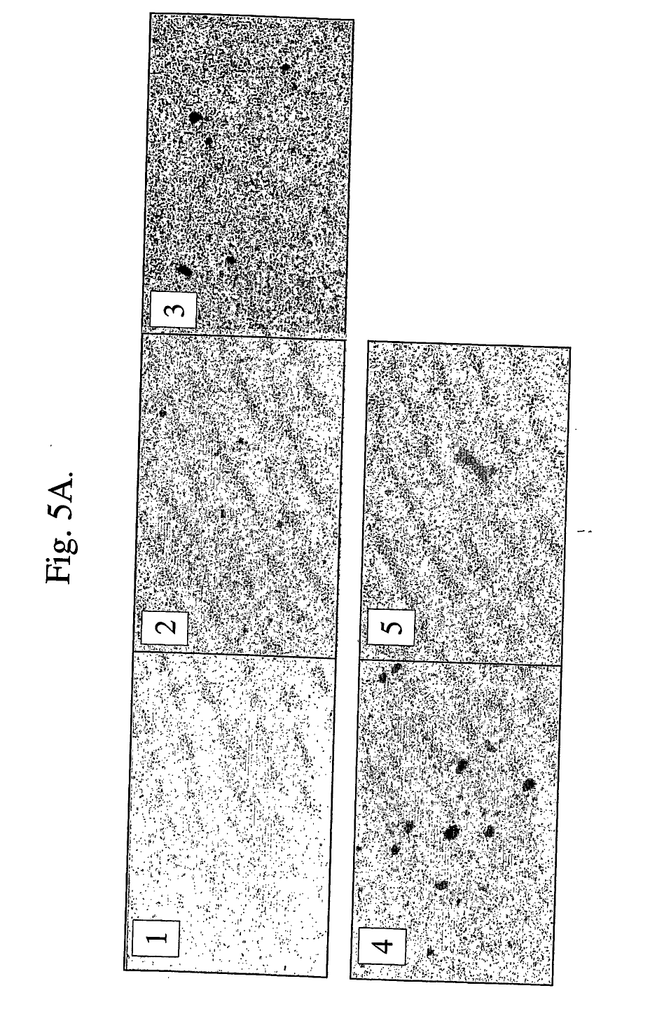

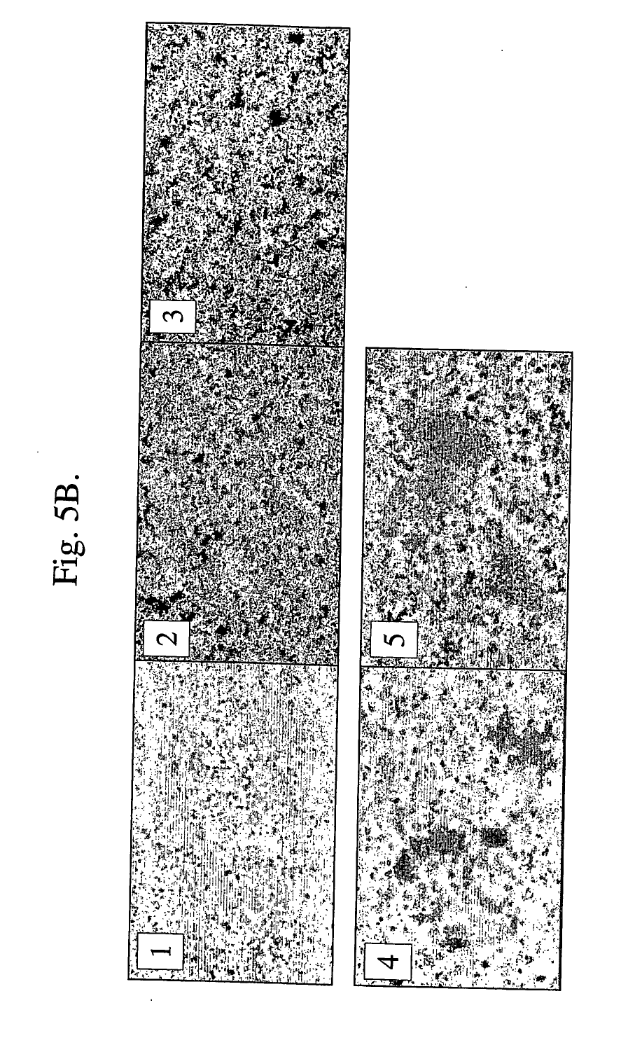

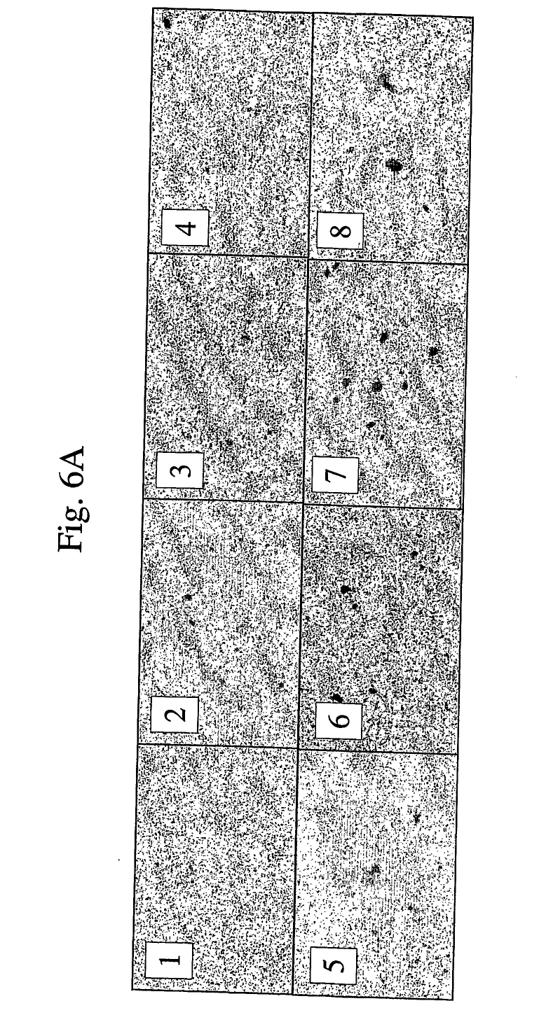

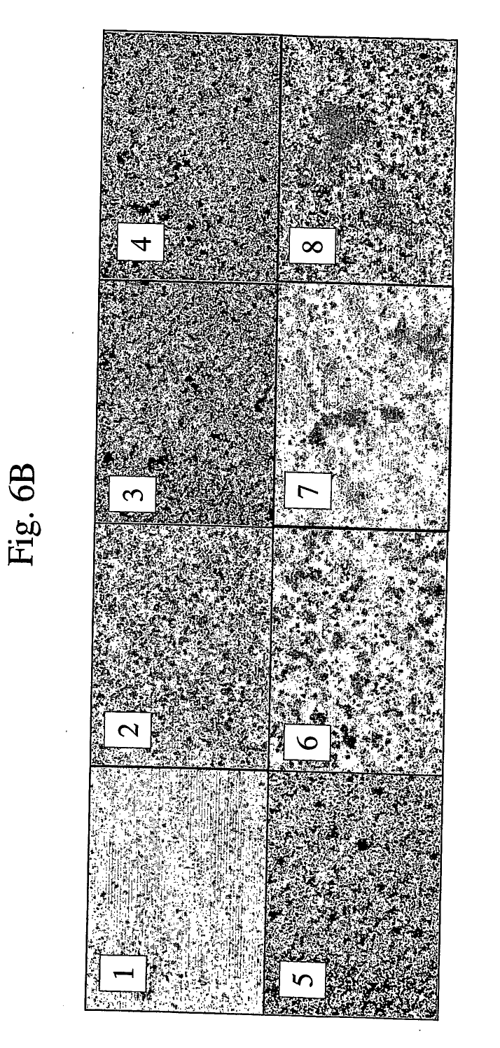

Latex Agglutination Test for Platelet Product. Addition of the platelet suspensions to latex beads renders the latex nearly invisible on black or white paper (FIG. 4). The latex-antibody detection test for platelets was therefore examined under the microscope using hanging-drop glass slides by using the 4+ scoring criteria (−, +, ++, +++, ++++) described above (FIG. 5). Latex-anti-Staphepi-IgG (1:5) detected Staphylococcus epidermidis, in platelet product, in a dose-dependent fashion (FIG. 6). Using latex-anti-Staphepi-IgG, we detected Staphylococcus epidermidis contamination in platelets as low as 1/16 of McFarland 1 Standard, within 5 minutes of reaction time. This is equivalent to a concentration of 2.5×10⁷ bacteria/ml. This establishes proof of principle that agglutination methods are sensitive enough to detect bacteria at a concentration heretofore unappreciated. It would be understood that greater sensitivity could be achieved using routine methods, for example by selecting for antibodies with the greatest affinity, or by using an instrument that measures light scattering.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference in their entireties. The discussion of the references herein is intended merely to summarize the assertions made by the authors and no admission is made that any reference constitutes prior art. Applicants reserve the right to challenge the accuracy and pertinence of the cited references.

1. A method of detecting a microorganism in an aqueous solution or suspension, the aqueous solution or suspension not comprising precultured microorganisms, the method comprising
mixing the solution or suspension with microspheres coated with antibodies or antibody fragments comprising an antigen binding site selective for the microorganism, to create a microsphere-solution/suspension mixture; then
evaluating the microsphere-solution/suspension mixture for agglutination,
wherein the presence of agglutination indicates that the solution or suspension contains the microorganism.

2. The method of claim 1, wherein the solution or suspension is obtained from a food, an animal feed, or a water supply.

3. (canceled)

4. The method of claim 1, wherein the solution or suspension is obtained from a mammal.

5. (canceled)

6. The method of claim 1, wherein the solution or suspension is obtained from a specimen selected from the group consisting of urine, stool, sputum, bronchial aspirate, cerebrospinal fluid, pus or blood.

7. The method of claim 6, wherein the solution or suspension is whole blood or comprises a blood product.

8. The method of claim 7, wherein the solution or suspension is plasma or serum.

9. The method of claim 7, wherein the solution or suspension comprises a blood product that is substantially purified from other blood products, the blood product selected from the group consisting of red blood cells, platelets, factor IX, factor VIII, albumin, and antibodies.

10. The method of claim 9, wherein the blood product is red blood cells or platelets.

11. The method of claim 9, wherein the blood product is platelets.

12. The method of claim 1, wherein the microorganism is a bacterium.

13-15. (canceled)

16. The method of claim 12, wherein the bacterium is selected from the group consisting of a Staphylococcus sp., a Pseudomonas sp., a Listeria sp., an Enterobacteriaceae species, a Vibrionaceae species, a Clostridium sp., a Campylobacter sp., a Bacillus sp., Escherichia coli, a Sarcina sp., a Flavobacterium sp., a Bacillus sp., an Alcaligenes sp., a Micrococcus sp., a Serratia sp., a Klebsiella spp., a Streptococcus sp., a Herellea sp., a Corynebacterium sp., a Mycoplasma sp., a Pseudomonas sp., a Citrobacter sp., a Treponema sp., a Salmonella sp., Serretia marcescens, Yersinia enterocolitica, a Legionella sp., a Bartonella sp., and a Brucella sp.

17. (canceled)

18. The method of claim 12, wherein the bacterium is Staphylococcus epidermidis.

19-28. (canceled)

29. The method of claim 1, wherein the microspheres are fluorescent.

30-33. (canceled)

34. The method of claim 1, wherein the antibodies or antibody fragments are fluorescent.

35. (canceled)

36. The method of claim 1, wherein the microsphere-solution/suspension mixture is evaluated on a hanging drop slide.

37. The method of claim 1, wherein the microsphere-solution/suspension mixture is evaluated with a microscope.

38. (canceled)

39. The method of claim 1, wherein the microsphere-solution/suspension mixture is evaluated with an instrument that measures light scattering.

40. (canceled)

41. The method of claim 1, wherein the microsphere-solution/suspension mixture is evaluated visually with the naked eye.

42. The method of claim 1, further comprising applying the microsphere-solution/suspension mixture to a gel surface then subjecting the gel with the microsphere-solution/suspension mixture to centrifugation before the evaluation step, wherein substantial retardation of movement of the microspheres after centrifugation when compared to the microspheres without the solution indicates the presence of agglutination.

43-58. (canceled)

59. A method of detecting at least one of at least n microorganism species in an aqueous solution or suspension, the method comprising
mixing the solution or suspension with microspheres coated with n distinct antibodies or antibody fragments comprising an antigen binding site, wherein each of the n distinct antibodies or antibody fragments comprising an antigen binding site is selective for the one of the n microorganism species, and wherein microspheres coated with antibodies or antibody fragments comprising an antigen binding site selective for each of the n microorganism species is present, to create a microsphere-solution/suspension mixture; then
evaluating the microsphere-solution/suspension mixture for agglutination,
wherein the presence of agglutination indicates that the solution or suspension contains at least one of the n microorganism species.

60-89. (canceled)
